(19) United States
(12) Patent Application Publication
(10) Pub. No.: US 2005/0142136 A1
(43) Pub. Date: Jun. 30, 2005

(54) Anti-interleukin 8 therapy for tumor osteolysis

(76) Inventors: Lary Suva, Little Rock, AR (US); Manali Bendre, Little Rock, AR (US)

Correspondence Address: Benjamin Aaron Adler;ADLER & ASSOCIATES, 8011 Candle Lane, Houston, TX, 77071, US

(21) Appl. No.: 10/972,834

(22) Filed: Oct. 25, 2004

Related U.S. Application Data
(60) Provisional application No. 60/513,903, filed on Oct. 23, 2003.

(51) Int. Cl. A61K 39/395

(52) U.S. Cl. 424/145.1; 514/2

(57) ABSTRACT

The present invention reports stimulatory effects of interleukin 8 (IL-8) on human osteoclast formation and bone resorption, indicating IL-8 as a potent activator of bone destruction common in metastatic bone diseases. Tumor growth and osteolysis were inhibited by anti-IL-8 antibody or antisense IL-8. Additionally, IL-8 was able to confer an osteolytic phenotype on non-osteolytic cancer cells. These results identify tumor-induced osteolysis and bone resorption as potential targets of anti-IL-8 therapy.

### CROSS REFERENCE TO RELATED APPLICATION

This non-provisional application claims benefit of provisional application U.S. Ser. No. 60/513,903 filed on Oct. 23, 2003, now abandoned.

### FEDERAL FUNDING LEGEND

This invention was produced in part using funds obtained through a grant (NIH RO1DK54044) from the National Institutes of Health. Consequently, the federal government has certain rights in this invention.

### BACKGROUND OF THE INVENTION

1. Field of the Invention

The present invention relates generally to the study of bone loss and cancer. More specifically, the present invention describes a role of interleukin-8 in the stimulation of osteoclast formation and tumor-induced bone destruction.

2. Description of the Related Art

Interleukin 8 (IL-8) is a member of the alpha chemokine family of cytokines that were originally identified as monocyte-derived factors capable of attracting and activating neutrophils. Numerous studies have revealed that IL-8 exhibits multiple functions in addition to its chemotactic activity. IL-8 receptors CXCR1 and CXCR2 have been identified on a number of cell types, including neutrophils, T lymphocytes, monocytes, endothelial cells and some tumor cell types, raising the possibility that IL-8 may have effects on a variety of cell types other than leukocytes. The role of IL-8 as a potent angiogenic agent is now well established. IL-8 has also been implicated in the progression of many tumor types due to its ability to enhance the proliferation, adhesion, invasion, angiogenesis and metastatic potential of tumor cells.

Human osteoclasts have been reported to synthesize IL-8, which has been suggested to act as a potential regulatory signal for cell recruitment during bone remodeling. In addition, IL-8 mRNA expression is also stimulated by murine macrophage colony stimulating factor (m-CSF) which supports the proliferation of osteoclast progenitors. IL-8 can also be secreted by a variety of other cells in the bone microenvironment, including bone marrow stromal cells, osteoblasts, osteoclasts, synovial fibroblasts, and chondrocytes. IL-8 expression has been shown to be elevated during a number of inflammatory processes like rheumatoid arthritis, osteoarthritis and osteomyelitis, which are associated with osteoclast activation and joint and bone destruction.

IL-8 is present at significant levels in the synovial fluid of patients with osteoarthritis, rheumatoid arthritis and temporomandibular joint disorders. Furthermore, elevated IL-8 levels have also been observed in patients with periodontitis. In these disorders, IL-8 is proposed to act a potent chemoattractant enhancing inflammatory cell infiltration and thus contributing to joint and bone destruction.

It was reported recently that there was elevated serum levels of IL-8 in patients with Cushing's syndrome, a disease characterized by osteoporosis and hypersecretion of the immunosuppressive and anti-inflammatory hormone cortisol from the adrenal glands. This disease is associated with reduced bone mass as well as area, most likely related to decreased bone formation and increased bone resorption via osteoclasts.

Another study investigating serum levels of multiple cytokines in patients with post-menopausal osteoporosis found elevated levels of IL-8, suggesting that IL-8 may play a role in the high bone turnover associated with post-menopausal osteoporosis. IL-8 has also been shown to up-regulate parathyroid hormone (PTH) production by cells of the parathyroid gland. Regulation of parathyroid hormone, a hormone involved in the normal calcium metabolism coupled with the production of IL-8 by a variety of different cell types in bone suggests that IL-8 may be involved in normal bone homeostasis.

The above evidences suggest that IL-8 has a role that goes far beyond the functions for which it was first identified. However, the precise role of IL-8 in pathological processes involving skeletal destruction, such as metastatic bone disease, or even in normal bone remodeling are largely undefined.

The prior art is lacking in an understanding of the role of interleukin-8 in the stimulation of osteoclast formation and bone resorption. The present invention fulfills this long-standing need and desire in the art.

### SUMMARY OF THE INVENTION

In the present study, the intriguing effect that IL-8 may stimulate osteoclastic bone resorption was demonstrated. The results indicate that IL-8 stimulated both osteoclastogenesis and bone resorption in human osteoclasts derived from peripheral blood mononuclear cells. The stimulatory activity of IL-8 was maintained even in the presence of excess receptor activator of NF kappa B (RANK)-Fc. In addition, IL-8 was also able to regulate the expression of the essential osteoclastogenic factor, receptor activator of NK-kB ligand (RANKL), by osteoblastic stromal cells. The role of IL-8 in bone destruction was confirmed by in vivo experiments in which anti-IL-8 antibody or antisense IL-8 blocked tumor growth and osteolysis. Additionally, in vivo experiments in the present study also demonstrated the ability of IL-8 to confer an osteolytic phenotype on non-osteolytic MDA-231 cells, thereby providing support to the hypothesis that IL-8 plays a role in the bone destruction associated with breast cancer and in the homing of tumor cells to bone.

This is the first report demonstrating a stimulatory effect of IL-8 on the process of human osteoclastogenesis and bone resorption. These data implicate IL-8 as a potent activator of the bone destruction common in metastatic bone disease. Taken together, these data directly link IL-8 with promoting osteolysis and provide a mechanistic basis for the role of IL-8 in the bone loss associated with metastatic cancer. Furthermore, these data also suggest that other diseases characterized by high bone turnover and bone loss may be related to the elevated serum levels and activities of IL-8.

The present invention is directed to a method of decreasing tumor growth and tumor-induced bone destruction in a subject. This method comprises the step of administering to the subject a compound that inhibits the binding of interleukin 8 (IL-8) to its receptor.

The present invention is further directed to a method of decreasing tumor growth and tumor-induced bone destruction in a subject. This method comprises the step of administering to the subject a compound that inhibits the expression of interleukin 8 (IL-8).

The present invention is also directed to a method of decreasing bone resorption in a subject. This method comprises the step of administering to the subject a compound that inhibits the binding of interleukin 8 (IL-8) to its receptor.

The present invention is further directed to a method of decreasing bone resorption in a subject. This method comprises the step of administering to the subject a compound that inhibits the expression of interleukin 8 (IL-8).

The present invention is also directed to a method of decreasing osteolytic activity of cancer cells in a subject. This method comprises administering to the subject a compound that inhibits binding of interleukin 8 (IL-8) to its receptor. This inhibits homing of the cancer cells to the bone, which decreases the osteolytic activity of the cancer cells in the subject.

The present invention is further directed to a method of decreasing osteolytic activity of cancer cells in a subject. This method comprises administering to the subject a compound that inhibits the expression of interleukin 8 (IL-8). This inhibits homing of the cancer cells to the bone, which decreases the osteolytic activity of the cancer cells in the subject.

Other and further aspects, features, and advantages of the present invention will be apparent from the following description of the presently preferred embodiments of the invention. These embodiments are given for the purpose of disclosure.

### BRIEF DESCRIPTION OF THE DRAWINGS

FIGS. 1A-B show expression of receptor activator of NK-kB ligand (RANKL) expression in osteoblastic cells in response to rhIL-8. FIG. 1A shows IL-8 increases RANKL mRNA production in MC3T3-E1 osteoblastic cells. Time course of RANKL and osteoprotegerin (OPG) expression in MC3T3-E1 cells in response to rhIL-8 was determined by RT-PCR. MC3T3-E1 cells were grown in 6-well tissue culture dishes until ˜80% confluence. Recombinant hIL-8 (10 ng/ml) was added to all wells except control. Cells were harvested at 2, 4, 6, 8, 10, 12 and 24 hours after addition of IL-8 for RNA extraction. Equal amounts of RNA were utilized in the RT-PCR reaction to estimate the relative expression of RANKL and osteoprotegerin in seven IL-8 treated and one control sample.

FIG. 1B shows immunofluorescence analysis for RANKL protein expression in MC3T3-E1 osteoblastic cells. MC3T3-E1 cells were cultured in 4-chamber well slides. At 80% confluence rhIL-8 (10 ng/ml) was added. Twenty-four hours after addition of rhIL-8, media was removed and cells were fixed in 10% formalin. Immunofluorescence analysis for RANKL was done on the IL-8 treated and untreated cells for comparison. The no primary antibody panel is the control well in which only control secondary antibody was added. Original magnification ×20.

FIGS. 2A-C compares TRAP⁺ multinucleated cell formation in human peripheral blood mononuclear cells in various treatment groups. In FIG. 2A, human peripheral blood mononuclear cells (0.5 million cells per well in 0.5 ml) were cultured in 48-well tissue culture plates. Murine macrophage colony stimulating factor (m-CSF) (25 ng/ml), RANKL (25 ng/ml), RANK-Fc (200 ng/ml) and rhIL-8 (10 ng/ml) were added to the respective wells (n=4 per treatment). On day 10, cultures were rinsed, fixed and stained for TRAP. TRAP⁺ multinucleated cells were observed in IL-8 or RANKL treated wells. Original magnification ×20. In FIGS. 2B-C, TRAP⁺ multinucleated cells (>3 TRAP⁺ MNC) formed were counted as mature osteoclasts. Results were expressed as mean (±SEM) number of osteoclasts per well per treatment (n=4 per treatment). Significance levels *p<0.01. The experiments were repeated two to three times with similar results.

FIG. 3 shows resorption area that was measured using Osteomeasure histomorphometry software after different treatments. Human peripheral blood mononuclear cells were cultured on dentine slices in the presence of murine macrophage colony stimulating factor (m-CSF) (25 ng/ml), rhIL-8 (10 ng/ml) and RANKL (25 ng/ml) (n=4 per treatment). On day 10, dentine slices were fixed and stained for TRAP. Slices treated with rhIL-8 or RANKL contained TRAP⁺ multinucleated cells resorbing dentine. Results were expressed as mean±SEM resorption area (sq. mm) per 8.64 mm measured per dentine slice (n =4 per slice). This experiment was repeated twice with similar results. *p<0.01.

FIGS. 4A-D show CXCR1 immunostaining of human osteoclasts. Human peripheral blood mononuclear cells were cultured at 37° C. in chamber well slides at a concentration of 1×10⁶ cells in the presence of RANKL (25 ng/ml) and murine macrophage colony stimulating factor (m-CSF) (25 ng/ml). FIGS. 4A and 4C show background (no primary antibody) staining of day 5 and day 10 cultured human osteoclasts using carboxyfluorescein-conjugated mouse IgG_{2A} isotype antibody. FIGS. 4B and 4D show specific CXCR1 immunofluorescent staining of day 5 (FIG. 4B) and day 10 (FIG. 4D) cultured human osteoclasts using FITC-conjugated anti-human CXCR1 antibody (15 ug/ml). Original magnification ×20.

FIG. 5 shows fluorescent microscopy showing double labeling of CXCR1 and the nuclei of human osteoclasts. Human peripheral blood mononuclear cells were cultured at 37° C. in chamber well slides at a concentration of 1 x 106 cells in the presence of RANKL (25 ng/ml) and murine macrophage colony stimulating factor (m-CSF) (25 ng/ml). Left panel shows background staining (carboxyfluorescein-conjugated mouse IgG_{2A} isotype control antibody) of day 10 cultured human osteoclasts. Right panel shows specific double staining of day 10 cultured human osteoclasts. CXCR1 detected in green (FITC-conjugated anti-human CXCR1 antibody 15 ug/ml) was present on many cells. Nuclei (blue, propidium iodide) were detected and numerous multi-nucleated cells were visible. Clear co-localization of multi-nucleated cells that are positive for CXCR1 were shown (arrows). Original magnification ×20.

FIGS. 6A-C show histology of IL-8 antibody experiment. Mice treated with IL-8 antibody showed small tumor foci (FIG. 6A) whereas IgG control antibody (FIG. 6B) and no antibody (FIG. 6C) treated mice had large tumors in their right legs. Extensive osteolytic bone destruction of the tibia was seen in mice treated with control antibody or no antibody.

FIGS. 7A-C show Micro CT reconstructions of mice treated with IL-8 antibody, control IgG and no antibody. Extensive osteolytic bone destruction was seen in mice treated with control antibody (FIG. 7B) or no antibody (FIG. 7C) compared to IL-8 antibody treated (FIG. 7A) group. Bar=1 mm.

FIGS. 8A-D show histomorphometric measurements performed on the tibiae of mice treated with either IL-8 antibody, IgG control or no antibody in order to quantitate tumor size and extent of osteolysis. FIG. 8A shows bone area (mm²) per total area (0.9 m m²). Values represent mean±SEM. *P<0.05. FIG. 8B shows tumor area (mm2) per total area (0.9mm²). Values represent mean±SEM. *P<0.05. FIG. 8C shows erosion perimeter (mm) per mm bone perimeter. Values represent mean±SEM. *P<0.05. FIG. 8D shows number of osteoclasts per mm bone perimeter. Values represent mean±SEM. *P<0.05.

FIGS. 9A-B show amount of IL-8 expressed in stably transfected MDA-MET and MDA 231 cells. FIG. 9A compares amount of IL-8 expressed per 10,000 cells in MDA-MET cells stably expressing IL-8 antisense cDNA to that in control transfected MDA-MET cells (MDA-MET Control TF). FIG. 9B compares amount of IL-8 expressed per 10,000 cells in MDA-231 cells (MDA-231-SI) stably transfected with IL-8 cDNA to that in untransfected MDA-231 cells.

FIGS. 10A-D show histomorphometry measurements performed on tibiae of mice injected with either MDA-MET cells stably transfected with IL-8 antisense cDNA (MDA-MET AS) or control transfected MDA-MET cells (MDA-MET Control TF). FIG. 10A shows bone area (mm²) per total area (0.9 mm²). Values represent mean±S.E. *P<0.05. FIG. 10B shows tumor area (mm²) per total area (0.9 m m²). Values represent mean±S.E. *P<0.05. FIG. 10C shows erosion perimeter (mm) per mm bone perimeter. Values represent mean±S.E. *P<0.05. FIG. 10D shows number of osteoclasts per mm of bone perimeter. Values represent mean±S.E. *P<0.05.

FIGS. 11A-B show Micro CT reconstructions of MDA-231 and MDA-231-S1 intratibial injections (4 weeks). Extensive osteolytic bone destruction of the tibia was observed in mice injected with IL-8 overexpressing cells, MDA-231-S1 (FIG. 1B) than with MDA-231 (FIG. 11A) cells.

FIG. 12 compares the osteoclast formation in MDA-MET conditioned media to that in MDA-231 conditioned media.

FIG. 13 shows schematic representation of the mechanism leading to amplification of osteolysis of breast cancer metastasis. PTHrP negative human breast cancer cells expressing increased IL-8 find their way to the bone microenvironment. Increased IL-8 levels directly activate the differentiation of osteoclast progenitors and bone resorption and indirectly increases RANKL expression by osteoblasts (and other stromal cells in the marrow). Once bone resorption has been activated by IL-8, growth factors such as TGF β and IGF-11 are released, stimulating PTHrP, IL-1 and IL-1 1 levels in stromal cells and osteoblasts. This action also stimulates the release of factors that support tumor proliferation in bone. These activities combine to amplify the osteolysis of breast cancer metastasis.

### DETAILED DESCRIPTION OF THE INVENTION

Data presented in the present invention demonstrate that recombinant human IL-8 can stimulate human osteoclastogenesis both dependent and independent of receptor activator of NK-kB ligand (RANKL). The addition of rhIL-8 to cultures of MC3T3-E1 osteoblastic cells increased both RANKL mRNA and protein expression, with no effect on osteoprotegerin (OPG) expression. This increase in the RANKL/OPG ratio by IL-8 would tip the balance in favor of enhanced osteoclast formation.

IL-8 is shown to be able to directly stimulate osteoclast formation from mononuclear cells in the presence of RANK-Fc. Human osteoclast precursors isolated from peripheral blood in the presence of RANK-Fc were differentiated in culture by rhIL-8. These cells were also capable of resorbing bone. The differentiated osteoclast phenotype of these cells appeared identical to that of cells treated with RANKL. These data suggest that rhIL-8 not only enhances osteoclast formation in culture, but may also have a stimulatory effect on the activity of mature osteoclasts analogous to that seen with RANKL.

The actions of IL-8 reported herein consist of one action that is indirect, involving a stimulatory effect on osteoblast RANKL expression, and one that is clearly direct, affecting the osteoclastogenic response of human osteoclast precursors. The identification of the specific IL-8 receptor (CXCR1), for which IL-8 is the only known ligand, on the surface of human osteoclast precursors and differentiated osteoclasts supports the direct effect of IL-8 on osteoclasts.

Human osteoclasts have been reported to synthesize IL-8, which has been suggested to act as a potential regulatory signal for cell recruitment during bone remodeling. In addition, IL-8 mRNA expression is also stimulated by murine macrophage colony stimulating factor (m-CSF) which supports the proliferation of osteoclast progenitors. Thus, it is hypothesized that in pathological states (such as bone metastasis) the availability of IL-8 secreted from invading tumor cells could increase osteoclastic bone resorption, and by increasing osteoclast motility provide an increased area of demineralized bone for subsequent tumor adhesion which significantly enhances the colonization of bone by tumor cells.

The data presented here indicate that IL-8 plays a larger role in the physiological and pathological processes of bone loss than previously thought, and identifies tumor induced osteolysis and bone resorption as potential targets of anti-IL-8 therapy. It now appears likely that in addition to its well-described role as a mediator of the inflammatory response, IL-8 can directly stimulate both osteoclastogenesis and osteoclast mediated bone destruction. This effect is presumably mediated by the expression of the IL-8 specific receptor (CXCR1) on human osteoclasts and their progenitors.

In the present invention, there is provided a method of using a compound that inhibits the binding of interleukin 8 (IL-8) to its receptor to decrease tumor growth and tumor-induced bone destruction in a subject such as an animal or a human. In general, inhibition of IL-8 receptor binding can be accomplished by an anti-IL-8 antibody or an antagonist of the IL-8 receptor. Preferably, the IL-8 specific receptor is CXCR1. Examples of IL-8 receptor antagonists include Toyama's T-614 (iguratimod) and compounds disclosed by Patent Number: WO0134141 as well as other known to a person having ordinary skill in this art.

In another embodiment of the present invention, there is provided a method of using a compound that inhibits the expression of IL-8 to decrease tumor growth and tumor-induced bone destruction in a subject such as an animal or a human. In general, the compound comprises an antisense IL-8 construct.

In yet another embodiment of the present invention, there is provided a method of decreasing bone resorption in a subject, comprising the step of administering to the subject a compound that inhibits the binding of interleukin 8 (IL-8) to its receptor. The compound may be an anti-IL-8 antibody or an antagonist of IL-8 receptor. In one aspect, the IL-8 receptor is CXCR1 and the subject is an animal or a human. Generally, the subject will likely but not necessarily have a disease of unwarranted bone resorption such as osteoporosis.

In still another embodiment of the present invention, there is provided a method of decreasing bone resorption in a subject, the method comprises the step of administering to the subject a compound that inhibits the expression of interleukin 8 (IL-8). Generally, the compound comprises an IL-8 antisense construct and the subject is an animal or a human. Generally, the subject will likely but not necessarily have a disease of unwarranted bone resorption such as osteoporosis.

In still another embodiment of the present invention, there is provided a method of decreasing osteolytic activity of cancer cells in a subject, comprising: administering to the subject a compound that inhibits binding of interleukin 8 (IL-8) to its receptor, and inhibiting homing of the cancer cells to the bone, thereby decreasing the osteolytic activity of the cancer cells in the subject. The compound may be an anti-IL-8 antibody or an antagonist of IL-8 receptor. Generally, the IL-8 receptor is CXCR1 and the subject is an animal or human. Additionally, the cancer cells are but may not be limited to breast cancer cells.

In still yet another embodiment of the present invention, there is provided a method of decreasing osteolytic activity of cancer cells in a subject, comprising: administering to the subject a compound that inhibits expression of interleukin 8 (IL-8), and inhibiting homing of the cancer cells to the bone, thereby decreasing the osteolytic activity of the cancer cells in the subject. Generally, the compound comprises an IL-8 antisense construct and the subject is an animal or a human. Additionally, the cancer cells are but may not be limited to breast cancer cells.

The following examples are given for the purpose of illustrating various embodiments of the invention and are not meant to limit the present invention in any fashion. The present examples, along with the methods, procedures, treatments, molecules, and specific compounds described herein are presently representative of preferred embodiments. One skilled in the art will appreciate readily that the present invention is well adapted to carry out the objects and obtain the ends and advantages mentioned, as well as those objects, ends and advantages inherent herein. Changes therein and other uses which are encompassed within the spirit of the invention as defined by the scope of the claims will occur to those skilled in the art.

### EXAMPLE 1

IL-8 Increases Receptor Activator of NK-kB Ligand (RANKL) Expression In Osteoblastic Cells

It is well recognized that the expression of RANKL by osteoblasts is essential for osteoclast formation and function. Therefore, this example examines the effect of IL-8 on RANKL expression in an osteoblastic cell line which is capable of supporting osteoclast differentiation.

Cell Line And Culture Conditions

MC3T3-E1 cells were obtained from ATCC. The cell line was maintained in a MEM,supplemented with 10% fetal bovine serum (FBS) at 37° C. in sterile culture dishes. As required, cells were subcultured by trypsinization in 5 mg/ml trypsin (Sigma) and 0.5 mmol/I EDTA in HBSS without calcium or magnesium in a laminar flow hood during their logarithmic phase of growth.

RT-PCR For RANKL And Osteoprotegerin (OPG)

Total RNA was extracted from the MC3T3-E1 cells using the Qiagen RNeasy Midi kit (Qiagen, Inc., Valencia, Calif.) according to the manufacturer's instructions. Extracted RNA was quantitated by spectrophotometry and examined visually by agarose gel electrophoresis.

Reverse transcriptase-PCR analysis was performed using specific mouse RANKL and mouse OPG primers. The 726 bp mouse RANKL product was amplified using the following sequences: 5′-AAGCTTTGGATCCTAACAGAATATCAG-3′ (SEQ ID NO. 1) and 5′-MGCTTCAGTCTATGTCCTGMCTT-3′ (SEQ ID NO. 2). The 257bp mouse OPG product was amplified using the following sequences: 5′-AAAGCACCCTGTAGAAAACA-3′ (SEQ ID NO. 3) and 5′-CCGTTTTATCCTCTCTACACTC-3′ (SEQ ID NO. 4). RNA was reverse transcribed at 48° C. for 45 minutes. Each cycle set of PCR used a denaturing step (94° C. for 30 seconds), annealing (55° C. for 30 seconds) and extension (72° C. for 30 seconds) and this was repeated for 30 cycles. A final extension was performed at 72° C. for 10 minutes. The level of increased RANKL mRNA expression was quantified and compared using ImageQuant software (Molecular Dynamics) after normalization to the signals for the osteoprotegerin (OPG) gene, which were unchanged.

Time-Course of RANKL Expression In MC3T3-E1 Cells In Response To rhIL-8

MC3T3-E1 cells were grown in 6-well tissue culture dishes (2 wells per time point) until ˜80% confluence. Recombinant human (rh) IL-8 was added at a concentration of 10 ng/ml to all treatment wells. Cells were harvested at 2, 4, 6, 8, 10, 12 and 24 hours after addition of rhIL-8 for RNA extraction. Equal amounts of RNA were utilized in the RT-PCR reaction to determine the relative expression of RANKL and osteoprotegerin (OPG).

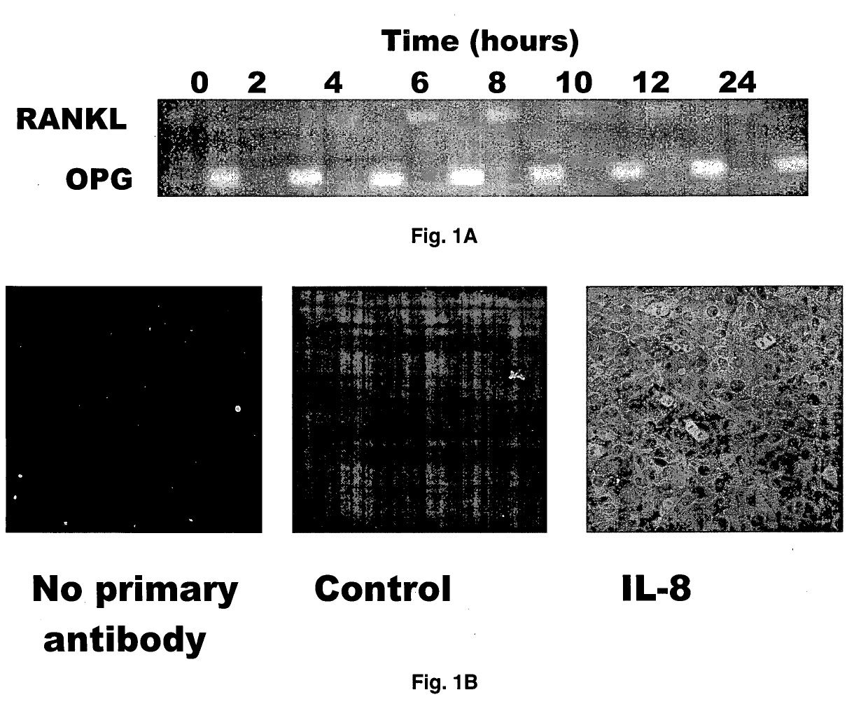

As shown in FIG. 1A, the addition of rhIL-8 (10 ng/ml) to cultures of murine MC3T3-E1 osteoblastic cells increased RANKL mRNA expression (2.6 fold), which peaked at 6 hours. However, osteoprotegerin levels remained unchanged over the same time period.

In addition, the same rhIL-8 treatment also induced RANKL protein expression as seen by immunofluorescence analysis (FIG. 1B). MC3T3-El cells were cultured in 4-well chamber slides (Lab-Tek, Nalgene Nunc International, Rochester, N.Y.). At ˜80% confluence, rhIL-8 was added to all treatment wells at a concentration of 10 ng/ml. Twenty-four hours after addition of rhIL-8, media was removed from the wells and the cells washed twice with PBS and fixed in 10% formalin at 4° C. for 30 minutes. After fixation, the cells were washed twice in PBS and blocked with 1% goat serum for 30 minutes at 37° C. The cells were then treated with goat anti-mouse RANKL antibody at a concentration of 10 ug/ml overnight at 4° C. The following day, the primary antibody was removed and the cells were washed 3 times with PBS. The washed cells were then blocked with 1% rabbit serum for 30 minutes at 37° C. FITC-conjugated rabbit anti-goat secondary antibody was added (1:300 dilution) and incubated for 1 hour at 37° C. The secondary antibody was then removed and the cells were washed 3 times with PBS. The wells were removed, and the slides were mounted in fluorescent mounting medium (Prolong Antifade Kit, Molecular Probes, Eugene, Oreg.). All slides were examined using a Olympus Fluoview microscope (Olympus America Inc., Melville, N.Y.). Representative images were taken with 20× and 40× objectives.

In summary, addition of IL-8 increased both RANKL mRNA and protein, thereby altering the RANKL/OPG ratio in the cells, in the favor of osteoclast formation. Similar levels of increased RANKL mRNA expression have been shown previously to support osteoclast formation by stromal cells.

### EXAMPLE 2

IL-8 Stimulates TRAP⁺ Multinucleated Cell Formation

This example examines whether IL-8 has a direct effect on osteoclast formation in human peripherial blood mononuclear cell cultures.

Peripheral blood was collected from healthy donors using heparin as an anticoagulant in the presence of 200 ng/ml RANK-Fc to minimize any priming of osteoclast progenitors by endogenous receptor activator of NK-kB ligand (RANKL). Blood was diluted in sterile PBS (1:1) in a sterile hood. The blood-PBS solution was slowly layered over Accu-Prep solution (Accurate Chemical and Scientific Corp., Westbury, N.Y.) and then centrifuged at 400 g in swing buckets for 30 minutes at 21° C. The peripheral blood mononuclear cell layer was collected and washed in 5-6 volumes of PBS, isolated by centrifugation at 140 g and re-suspended in aMEM containing 10% fetal bovine serum. Cells were counted with a hemocytometer and plated in 48-well tissue culture plates at a concentration of 0.5 million cells in 0.5 ml volume per well. Macrophage colony stimulating factor (mCSF; 25 ng/ml) was present in all treatment groups including control. RANKL (25 ng/ml), rhIL-8 (10 ng/ml), RANK-Fc (200 ng/ml), RANK-Fc+IL-8, RANKL+RANK-Fc and RANKL+IL-8 were used as treatments and were added to respective wells (n=4 per treatment). Cultures were maintained at 37° C. and half feeds were done three times per week, and terminated on the 10^{th} day. Media was aspirated and the cells fixed with 10% formalin. Tartrate resistant acid phosphatase (TRAP) staining was performed for quantitation of TRAP⁺ multinucelated cells (MNCs) as described previously (Gaddy-Kurten et al., 2002). TRAP⁺ cells having more than 3 nuclei were counted in the entire well with 4 wells per treatment. Cell counts were averaged and the results expressed as the number of TRAP⁺ MNCs/well per treatment group.

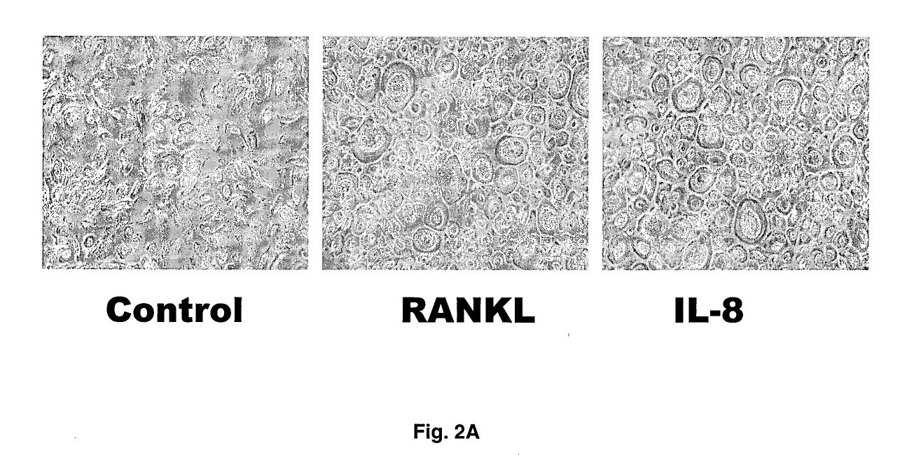

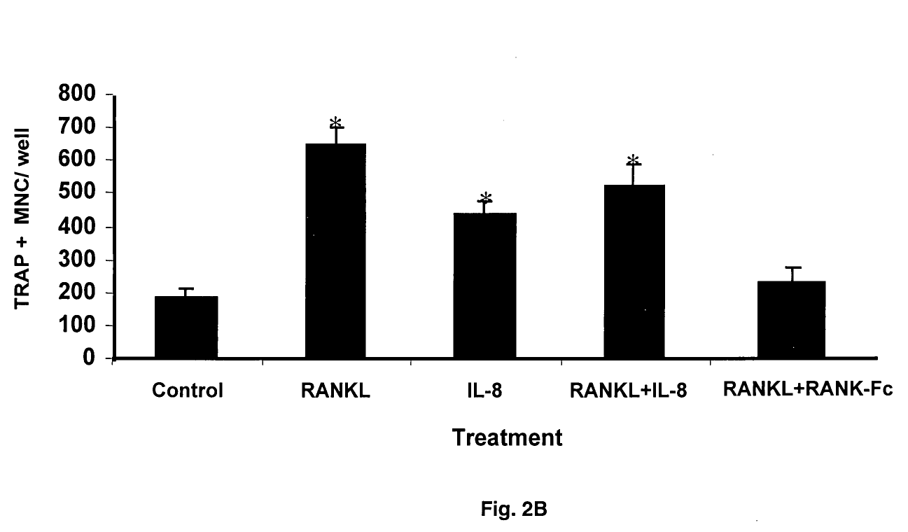

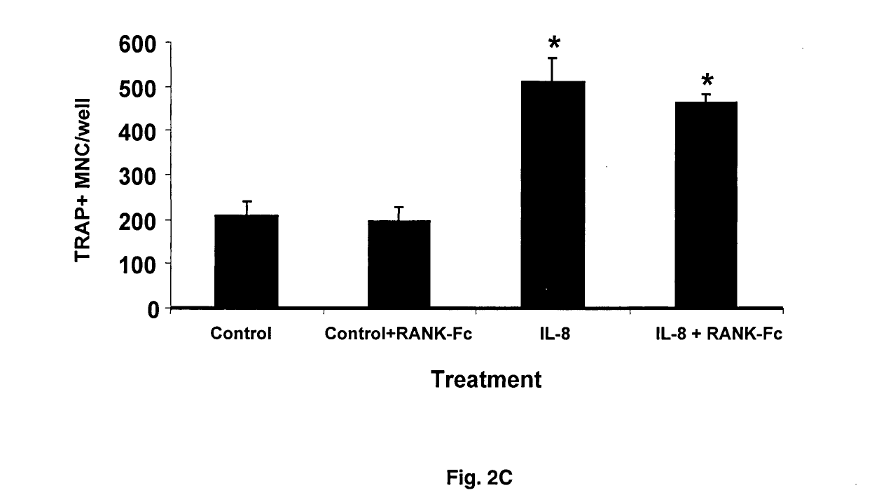

As shown in FIG. 2A, IL-8 stimulated TRAP⁺ multinucleated cell formation within 10 days even in the absence of exogenous RANKL. The number of TRAP⁺ multinucleated cells induced by IL-8 (10 ng/ml) was comparable to that seen with RANKL (25 ng/mi) (FIG. 2B). However, no additive or synergistic effects were seen when IL-8 (10 ng/ml) and RANKL (25 ng/ml) were added together. In addition, the ability of IL-8 (10 ng/ml) to stimulate osteoclast formation was not affected by the addition of 200 ng/ml RANK-Fc (FIG. 2C). In contrast, the same dose of RANK-Fc suppressed RANKL-stimulated osteoclast formation to basal levels (FIG. 2B). Basal levels of osteoclast formation in cultures containing only murine macrophage colony stimulating factor (m-CSF) as control were not diminished by the addition of RANK-Fc (FIG. 2C), suggesting that basal osteoclast formation in these cultures is the result of in vivo RANKL priming of osteoclast progenitors in peripheral blood.

The ability of IL-8 to stimulate TRAP⁺ multinucleated cell formation independent of RANKL was somewhat surprising. These data prompted a further evaluation of the osteoclastogenic effects of rhIL-8.

### EXAMPLE 3

IL-8 Stimulates Osteoclast Formation and Activity

In order to investigate the effect of rhIL-8 on osteoclast activity, rhIL-8 was added to human peripheral blood mononuclear cells cultured on dentine slices in the presence or absence of receptor activator of NK-kB ligand (RANKL).

Peripheral blood was collected from healthy donors and peripheral blood mononuclear cells were isolated as previously described. Dentine (from Professor Tim Skerry, London, UK) was sliced into 0.5×0.5 cm pieces. The slices were collected in H₂O, and sonicated twice for 1 minute each to remove particle debris. They were then rinsed in 2 changes of water in between sonication and sterilized for at least 30 minutes in 100% ethanol. All dentine slices were stored in 100% ethanol until use. On the day of culture, dentine slices were washed 4 times with PBS and twice with aMEM. Using sterile forceps, one slice was placed in each well of a 48-well plate containing 0.5 ml of aMEM and the plate was incubated at 37° C. for 30 minutes. Equilibration media was then aspirated off and PBMCs were added to the wells at a concentration of 1.0×10⁶ cells/well in 0.5 ml volume. Precursors were allowed to adhere to the slices for 4 hours at 37° C. Appropriate amounts of treatment media were prepared and 0.5 ml was added to the wells in a replicate 48-well plate (lacking dentine slices) with 4 wells per treatment group. Macrophage colony stimulating factor (25 ng/ml) was present in all treatment groups including control. The concentrations of RANKL and rhIL-8 were 25 ng/ml and 10 ng/ml respectively. Using sterile forceps, slices (with adherent cells) were transferred to the second 48-well plate, being careful not to invert the slices. Half the media was exchanged three times per week. Cells were allowed to grow on dentine slices for 10-12 days, after which time the cultures were terminated. Dentine slices were fixed in 10% formalin and stained for TRAP. The dentine slices were then mounted on glass slides and examined under a microscope. TRAP⁺ multinucleated cells showing ability to resorb bone were counted as osteoclasts. Bone resorption area was measured using histomorphometry software (Osteomeasure, Atlanta, Ga.) after removal of the cells by sonication.

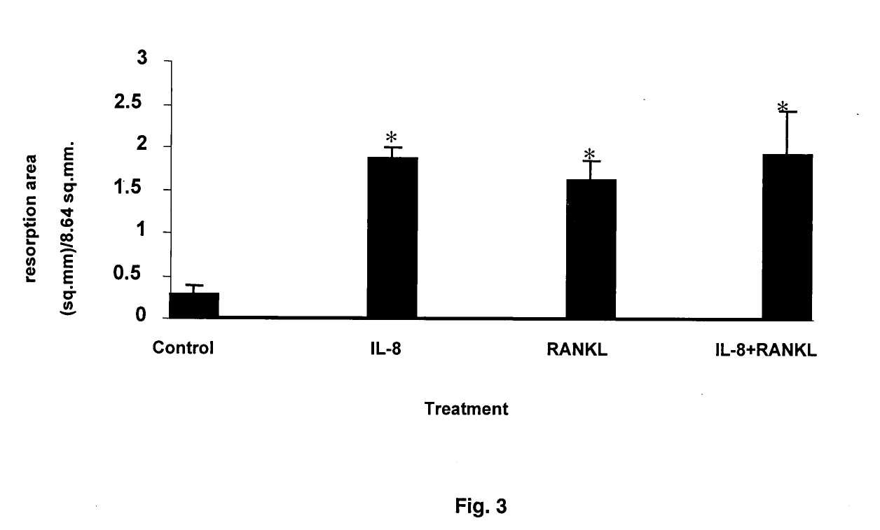

As shown in FIG. 3, TRAP positive multinucleated cells formed after 10 days of culture were able to resorb bone. Both osteoclast resorption lacunae and resorption trails due to both osteoclastic bone resorption and motility were observed in cultures treated with RANKL (data not shown). Similarly, treatment of peripheral blood mononuclear cells harvested in the presence of RANK-Fc, and cultured in the presence of rhIL-8 (10 ng/ml) and the absence of exogenous RANKL also stimulated osteoclastic bone resorption (FIG. 3). Thus, IL-8 was able to induce the formation of TRAP⁺ multinucleated cells that were capable of bone resorption in the absence of exogenous RANKL. The number, morphology and area of resorption by osteoclasts formed by IL-8 and by RANKL were comparable (FIG. 3). There were no apparent additive or synergistic effects on osteoclast number or bone resorption area when IL-8 and RANKL were added together at the concentrations used in this experiment.

### EXAMPLE 4

Human Osteoclast Precursors and Mature Osteoclasts Express IL-8 Receptor CXCR1

Having demonstrated that rhIL-8 directly influenced osteoclast formation in the presence of RANK-Fc in peripheral blood mononuclear cell cultures, this example investigates which of the receptors that bind IL-8 (CXCR1 and CXCR2) were expressed on osteoclast progenitors and mature osteoclasts.

Human peripheral blood mononuclear cells were cultured at 37° C. in chamber well slides at a concentration of 1×10⁶ cells in 1 ml media in the presence of RANKL (25 ng/ml) and murine macrophage colony stimulating factor (m-CSF) (25 ng/ml) as described above. Cultures were maintained as described above and half feeds performed every alternate day, with cultures terminated on either day 5 or day 10. The media was removed and the cells washed twice with PBS and fixed in 10% formalin for 30 minutes at 40 C. The cells were then washed twice with PBS and blocked for the primary antibody with 1% mouse serum for 30 minutes at 370 C. The cells were then stained overnight with FITC-conjugated anti-human CXCR1 antibody (15 ug/ml) at 4° C. Carboxyfluorescein-conjugated mouse IgG_{2A} isotype antibody was used as a control. The cells were then washed 3 times with PBS (5 minutes each) and then mounted in fluorescent mounting medium (Prolong Antifade). Propidium iodide was used to stain the nuclei in some cases before mounting and subsequent immunostaining. Cells were observed and photographed using a fluorescent microscope (Olympus Fluoview) at 20× magnification.

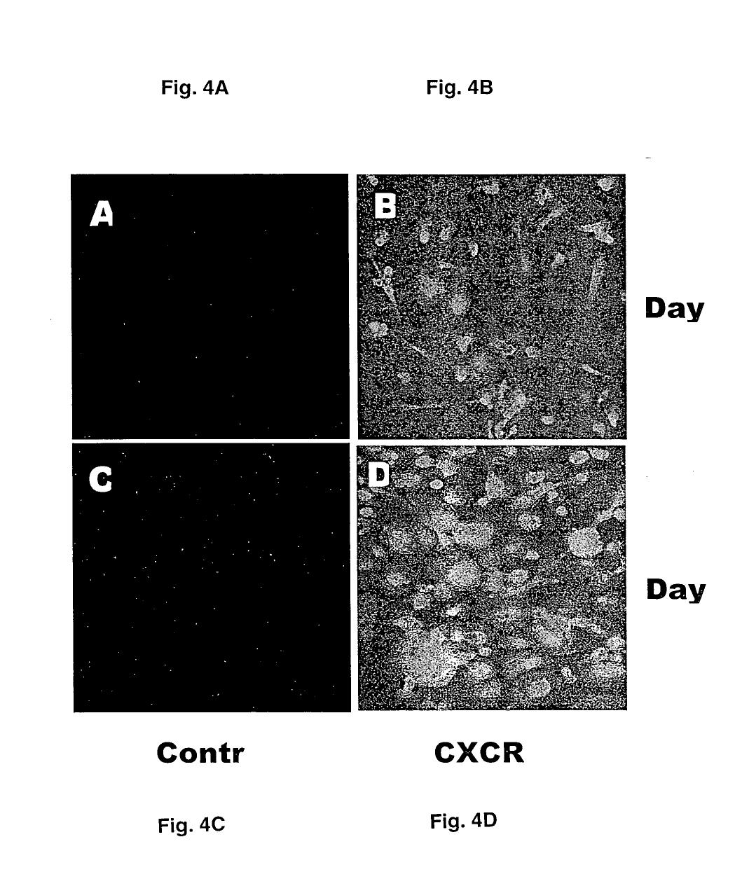

As shown in FIG. 4, five-day peripheral blood mononuclear cell cultures (containing osteoclast progenitors) and 10 day cultures (containing mature multinucleated osteoclasts) showed positive staining with FITC-conjugated antibody to CXCR1, the receptor for which IL-8 is the only know ligand (FIGS. 4B, D). In contrast, no specific staining was observed for CXCR2 (data not shown), which binds IL-8 as well as other ligands such as gro1-alpha.

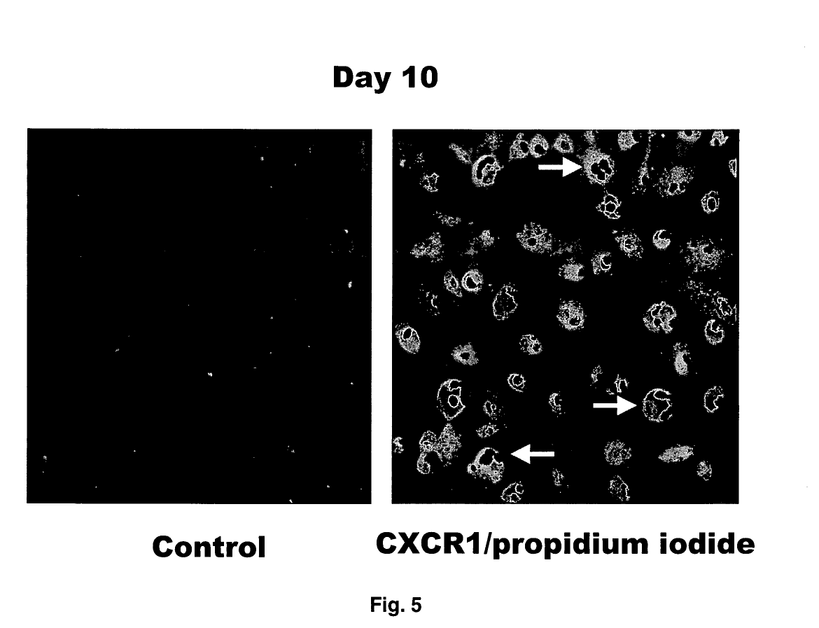

The CXCR1 immunostaining was confirmed on osteoclasts by double staining for cell nuclei (using propidium iodide) and CXCR1. This approach demonstrated numerous multinucleated, CXCR1 positive cells (FIG. 5), which were positive for alpha v beta 3 integrin, another marker of the osteoclast phenotype (data not shown). As expected in day 10 cultures of PBMC's, many mononucleated cells were also positive for CXCR1 (FIG. 5).

### EXAMPLE 5

Anti-IL-8 Antibody Inhibits Tumor Growth and Bone Resorption

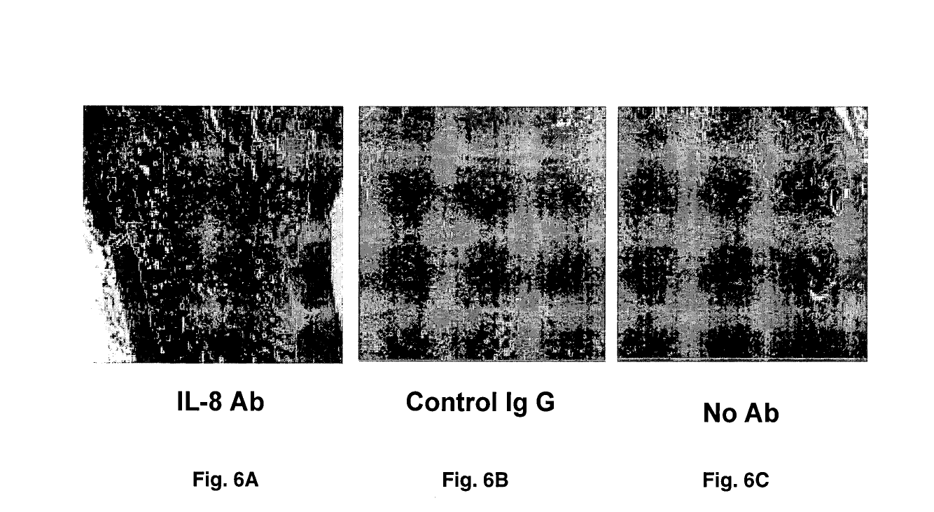

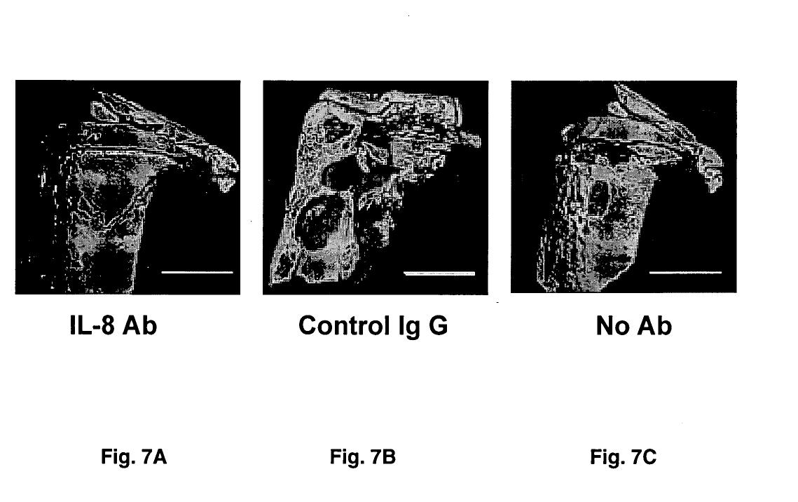

Since it was demonstrated that IL-8 could stimulate osteoclastogenesis and bone resorption, the effect of blockade of IL-8 in vivo on tumor osteolysis was further examined. This was accomplished by examining the anti-tumor efficacy of an anti-IL-8 neutralizing antibody on direct intratibial injection of MDA-MET cells. Briefly, nude mice were intratibially injected with 10,000 MDA-MET cells and treated with either a monoclonal antibody directed against IL-8 (35 mg) or an isotype control IgG (35 ug) or no treatment every alternate day for 4 weeks following tumor cell inoculation. Mice were then sacrificed and both legs evaluated by X-Ray, micro CT and histology. All animals receiving no treatment (6/6) or those treated with control IgG (6/6) developed large osteolytic bone tumors (FIGS. 6B and 6C). In contrast, 4/6 animals in the IL-8 antibody treated group had no evidence of tumor with the remaining two demonstrating only small tumor foci (FIG. 6A). However, it is likely that the small tumors observed are the result of sub-optimal antibody concentration in these animals. Further, the Micro CT evaluation of representative specimens (FIG. 7A-C) demonstrated bone destruction representative of the three treatment groups.

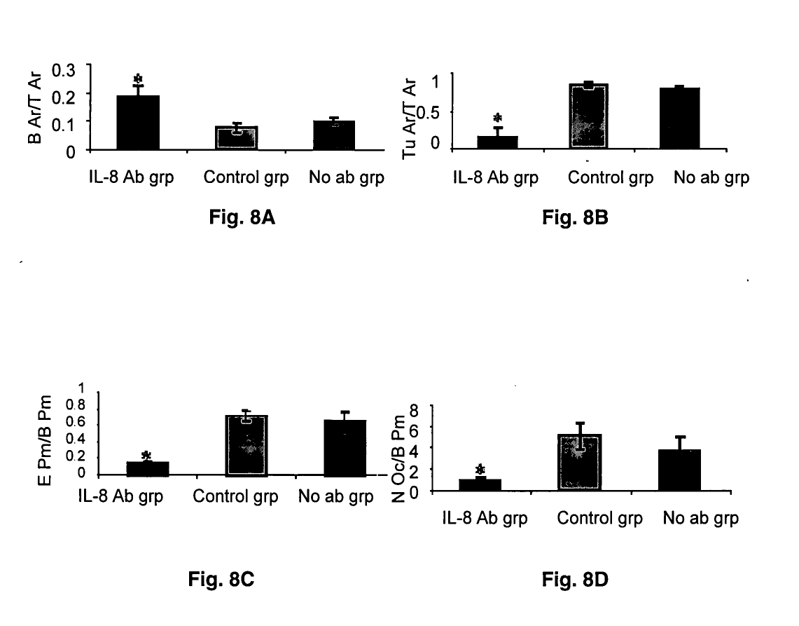

Additionally, quantitative histomorphometric measurements (Suva 1993) were performed on the tibiae of mice treated with either IL-8 antibody, IgG control or no antibody group in order to quantitate tumor size as well as the extent of osteolysis. The IL-8 antibody treated group had smaller tumors and more bone remaining than either the control IgG or no antibody groups (FIGS. 8A and 8B). The higher bone remaining in the IL-8 antibody group compared with the other groups was due to significantly decreased eroded surface and osteoclast number (FIGS. 8C and 8D). These data demonstrate that treatment with an IL-8 antibody significantly decreased tumor development, osteoclast formation and bone destruction.

### EXAMPLE 6

Antisense IL-8 Inhibits Tumor Growth and Bone Resorption

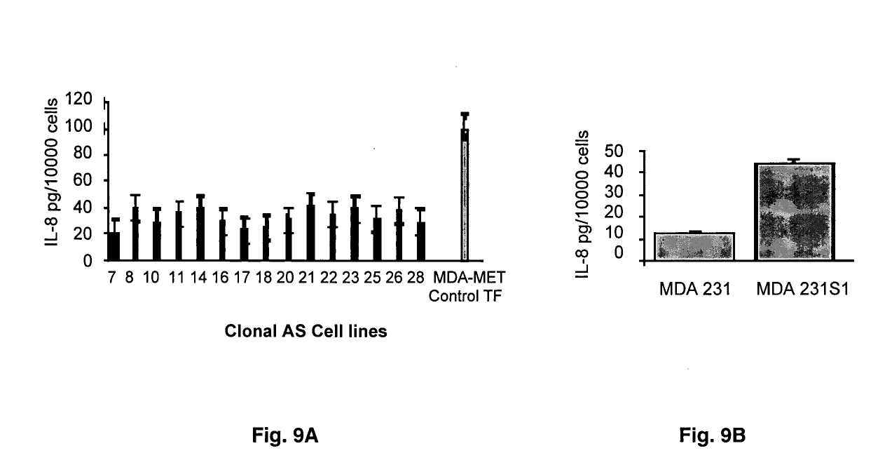

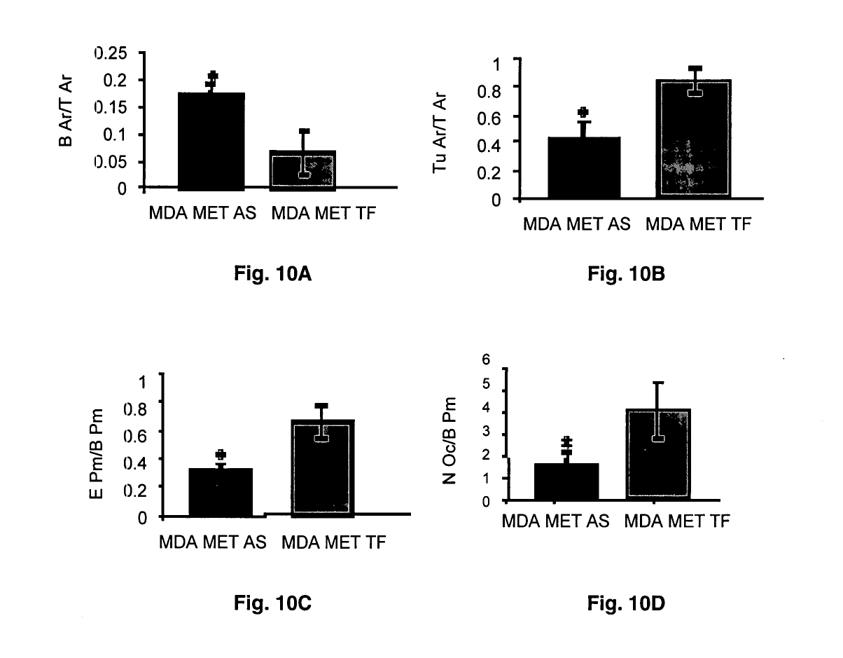

Next, IL-8 antisense cDNA was stably expressed in MDA-MET cells and its effect on tumor growth and bone resorption was examined. The MDA-MET cells stably expressing IL-8 antisense cDNA (MET AS 7) had significantly decreased IL-8 expression compared to control transfected MDA-MET cells, for example ˜20 pg/10,000 cells (FIG. 9A). Of the several clones that were obtained after transfection, Clone 7 was tested in vivo. Intratibial injection of these cells into nude mice produced smaller tumors with significantly lower osteoclast numbers and less bone destruction compared to mice injected with control transfected MDA-MET cells. The histomorphometric measurements were obtained from tibiae of nude mice injected with either MDA-MET AS or control transfected MDA-MET cells (FIGS. 10A-D).

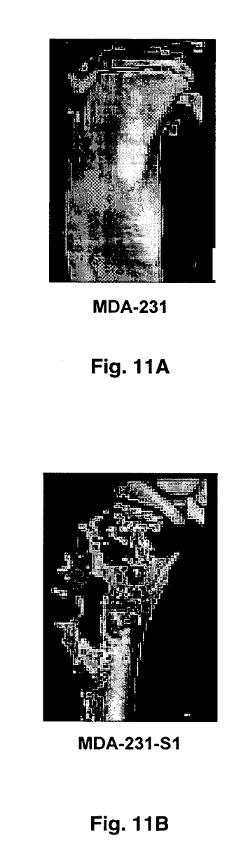

Since it was observed that blockade of IL-8 activity decreased tumor development, osteoclast formation and bone destruction, the effect of IL-8 overexpression on the osteolytic activity of non-osteolytic breast cancer cells in vivo was examined. The non-osteolytic MDA-231 cells were stably transfected with IL-8 cDNA and IL-8 expression in these cells was compared to that of untransfected MDA-231 cells (FIG. 9B). The stably transfected MDA-231S1 cells had increased IL-8 expression compared to untransfected MDA-231 cells. The stably transfected cells and untransfected cells were then injected directly into the tibia of nude mice and their effects on osteolysis compared. It was observed that untransfected MDA-231 cells did not show any evidence of osteolysis 4 weeks following injection (FIG. 11A). In distinct contrast, stable over-expression of high levels of IL-8 in MDA-231 cells (MDA 231S1) induced osteolytic bone destruction (FIG. 11B). These gain-of-function data demonstrate unequivocally that IL-8 confers an osteolytic phenotype on non-osteolytic MDA-231 cells.

Based on this data, it is contemplated that IL-8 plays an important role in the bone destruction associated with breast cancer and in the homing of tumor cells to the bone. Additionally, it also emphasizes the importance of anti-IL-8 approaches for treatment of breast cancer growth in bone and the functional evaluation of the role of IL-8 in tumor osteolysis in vivo.

IL-8 Secreted by MDA-MET Cells Induces Osteoclast Formation In Vitro

The results of in vivo experiments of the present study demonstrated that intratibial injection of MDA-MET cells produced large tumors with extensive bone destruction (FIG. 7) as opposed to intratibial injection of MDA-231 cells that did not (FIG. 11). Additionally, it was also observed that blockade of IL-8 levels using either a neutralizing antibody or transfection with IL-8 antisense significantly reduced tumor induced osteolysis in vivo (FIGS. 8, 10). Therefore, these results prompted evaluation of IL-8 secreted by MDA-231 and MDA-MET cells on human osteoclast formation in vitro.

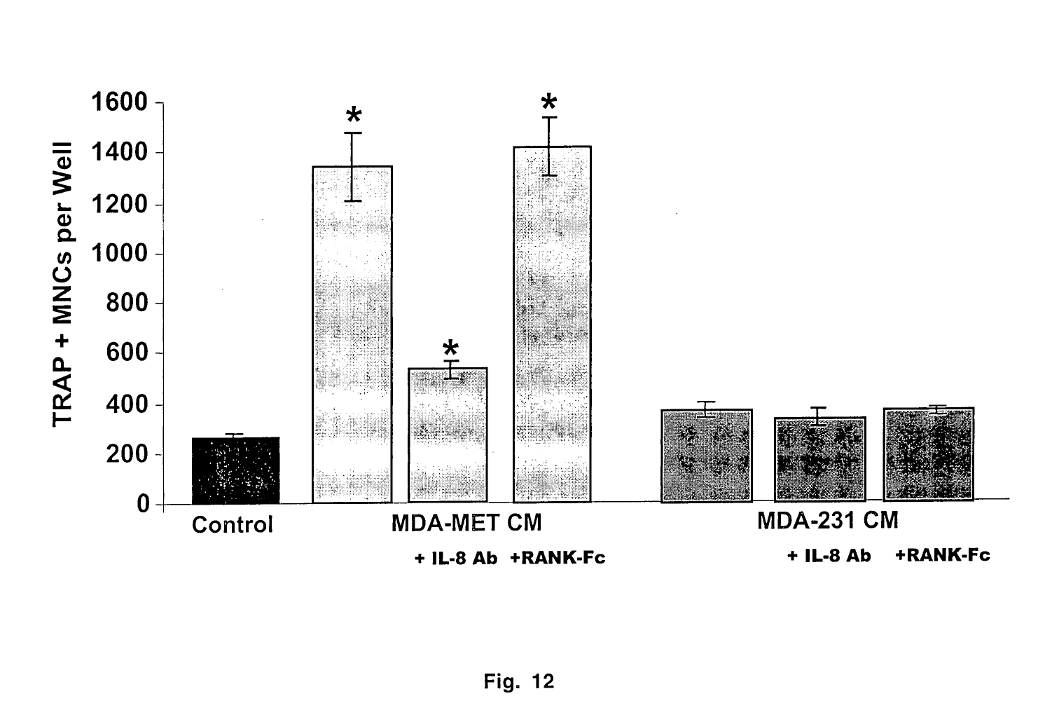

It is known that IL-8 (1-77) and IL-8 (6-77) are the major forms derived from endothelial cells or fibroblasts and leukocytes (Van den Steen, 2000 #6649, please provide details). To investigate the activity of IL-8 secreted by MDA-MET and MDA-231 cells, forty-eight hour conditioned media (containing serum) from MDA-231 and MDA-MET cells was collected and added to cultures of human peripheral blood mononuclear cells containing only mCSF (Bendre, 2003 #6760, please provide details). MDA-MET conditioned media stimulated osteoclast formation, whereas MDA-231 conditioned media did not (FIG. 12 (which is FIG. 17 in the document provided); please provide this figure).

Additionally, the activity of MDA-MET conditioned media was significantly but not completely inhibited with an IL-8 neutralizing antibody (FIG. 12). These data suggest that IL-8 is the major osteoclastogenic factor secreted by MDA-MET cells. However, MDA-MET cells also secrete factor(s) that can stimulate osteoclast formation, independent of RANKL. These data further emphasize the importance of determining the exact identity of the IL-8 isoform expressed by MDA-MET cells, which when identified will explain the increased osteolytic capacity discussed earlier.

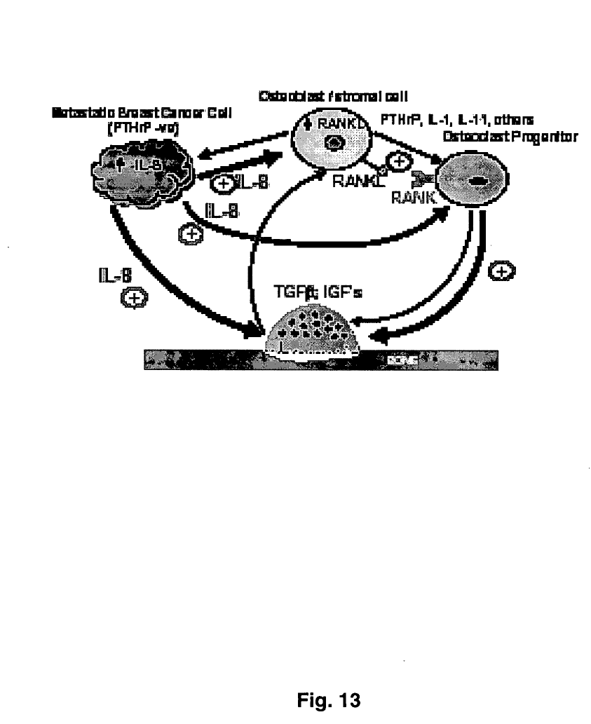

Taken together, the results of the present study provide evidence that strongly supports the feasibility of the approach taken to evaluate the novel role of IL-8 in the osteolytic phenotype of human breast cancer cells. Importantly, by examining the function of IL-8 in breast cancer, the present study for the first time demonstrates a correlation between IL-8 expression by breast cancer and tumor growth in bone. The direct regulation of osteoclastogenesis by IL-8, independent of RANKL, supports the contention that factors other than PTHrP expression (and its regulation by TGF-β) explain the phenotypic difference between MDA-231 and MDA-20 cells (FIG. 13).

In fact, PTHrP can only indirectly regulate osteoclast formation via upregulation of RANKL in stromal cells and has no reported direct effects on osteoclasts. It is more likely that factors (such as IL-8) increase bone resorption (by multiple mechanisms) that eventually lead to increased release of bone-derived growth factors that enhance bone resorption. Increased bone resorption then provides the stimulus for altering the bone marrow microenvironment in favor of a change in tumor phenotype such as the induction of PTHrP expression.

It is contemplated that the in vitro and in vivo assays of the present study will be useful in assessing the in vivo osteolytic phenotype of human breast cancer cells and in evaluating potential mediators of bone destruction and tumor growth. The data obtained in the present invention will also be extended to evaluate the mechanism by which IL-8 mediates increases in tumor growth and bone destruction as well as IL-8-gain and loss-of-function in vivo. Additionally it is also contemplated that the role of IL-8 in facilitating the ability of breast cancer cells to colonize and destroy the skeleton will also be delineated.

The following references were cited herein:
Bendre, Bone. 33(1): 28-37, 2003.
Gaddy-Kurten et al., Endocrinology 143:74-83 (2002).
Suva L. J., J. G. Seedor, N. Endo, H. A. Quartuccio, D. D. Thompson, l. Bab and G. A. Rodan. Pattern of gene expression following rat tibial marrow ablation. J Bone Miner Res. 8(3): 379-88., 1993.
Van den Steen, Blood. 96(8): 2673-81., 2000.

Any patents or publications mentioned in this specification are indicative of the levels of those skilled in the art to which the invention pertains. Further, these patents and publications are incorporated by reference herein to the same extent as if each individual publication was specifically and individually indicated to be incorporated by reference.

1. A method of decreasing tumor growth and tumor-induced bone destruction in a subject, comprising the step of administering to said subject a compound that inhibits the binding of interleukin 8 (IL-8) to its receptor.

2. The method of claim 1, wherein said compound is an anti-IL-8 antibody or an antagonist of IL-8 receptor.

3. The method of claim 2, wherein said IL-8 receptor is CXCR1.

4. The method of claim 1, wherein said subject is an animal or a human.

5. A method of decreasing tumor growth and tumor-induced bone destruction in a subject, said method comprises the step of administering to said subject a compound that inhibits the expression of interleukin 8 (IL-8).

6. The method of claim 5, wherein said compound comprises an IL-8 antisense construct.

7. The method of claim 5, wherein said subject is an animal or a human.

8. A method of decreasing bone resorption in a subject, comprising the step of administering to said subject a compound that inhibits the binding of interleukin 8 (IL-8) to its receptor.

9. The method of claim 8, wherein said compound is an anti-IL-8 antibody or an antagonist of IL-8 receptor.

10. The method of claim 9, wherein said IL-8 receptor is CXCR1.

11. The method of claim 8, wherein said subject is an animal or a human.

12. The method of claim 8, wherein said subject has osteoporosis.

13. A method of decreasing bone resorption in a subject, said method comprises the step of administering to said subject a compound that inhibits the expression of interleukin 8 (IL-8).

14. The method of claim 13, wherein said compound comprises an IL-8 antisense construct.

15. The method of claim 13, wherein said subject is an animal or a human.

16. The method of claim 13, wherein said subject has osteoporosis.

17. A method of decreasing osteolytic activity of cancer cells in a subject, comprising:
administering to said subject a compound that inhibits binding of interleukin 8 (IL-8) to its receptor; and
inhibiting homing of said cancer cells to the bone, thereby decreasing the osteolytic activity of said cancer cells in said subject.

18. The method of claim 17, wherein said compound is an anti-IL-8 antibody or an antagonist of IL-8 receptor.

19. The method of claim 18, wherein said IL-8 receptor is CXCR1.

20. The method of claim 17, wherein said subject is an animal or a human.

21. The method of claim 17, wherein said cancer cells are breast cancer cells.

22. A method of decreasing osteolytic activity of cancer cells in a subject, comprising:
administering to said subject a compound that inhibits expression of interleukin 8 (IL-8); and
inhibiting homing of said cancer cells to the bone, thereby decreasing osteolytic activity of said cancer cells in said subject.

23. The method of claim 22, wherein said compound comprises an IL-8 antisense construct.

24. The method of claim 22, wherein said subject is an animal or a human.

25. The method of claim 22, wherein said cancer cells are breast cancer cells.


SEQUENCE LISTING (4 sequences)

SEQ ID NO: 1 - DNA, 27 bp, Artificial Sequence
  primer_bind: primer for RANKL
aagctttggatcctaacagaatatcag

SEQ ID NO: 2 - DNA, 25 bp, Artificial Sequence
  primer_bind: primer for RANKL
aagcttcagtctatgtcctgaactt

SEQ ID NO: 3 - DNA, 20 bp, Artificial Sequence
  primer_bind: primer for OPG
aaagcaccctgtagaaaaca

SEQ ID NO: 4 - DNA, 22 bp, Artificial Sequence
  primer_bind: primer for OPG
ccgttttatcctctctacactc